(19) United States
(12) Patent Application Publication
(10) Pub. No.: US 2008/0104774 A1
(43) Pub. Date: May 8, 2008

(54) AMMONIA-FREE OXIDATION DYE FOR DYEING KERATIN FIBERS WITH ATMOSPHERIC OSYGEN SERVING AS THE SOLE OXIDIZING AGENT

(76) Inventors: Sabine Babiel, Moers (DE); Horst Hoffkes, Dusseldorf (DE); Marc Krippahl, Monchengladbach (DE); Claudia Kolonko, Remscheid (DE)

Correspondence Address: PAUL & PAUL, 2000 MARKET STREET, PHILADELPHIA, PA, 19103-3229, US

(21) Appl. No.: 11/853,318

(22) Filed: Sep. 11, 2007

Related U.S. Application Data
(63) Continuation of application No. PCT/EP2006/001151, filed on Feb. 9, 2006.

(30) Foreign Application Priority Data
Mar. 18, 2005 (DE) 10 2005 067.4

(51) Int. Cl. A61K 8/49; A61Q 5/10

(52) U.S. Cl. 8/409

(57) ABSTRACT

Agents for dyeing keratin fibers induced by atmospheric oxygen. These agents contain at least one dye precursor for a nature-analogous dye selected from the group consisting of indole derivatives or indoline derivatives, at least one oxidation dyestuff initial product of the developer type, and at least one oxidation dye initial product of the coupler type, and do not contain ammonia or additional oxidizing agents. The dyed keratin fibers obtained by using these agents exhibit intense, long-lasting color and do not have any red, blue or purple cast.

### CROSS-REFERENCE TO RELATED APPLICATIONS

This application is a continuation under 35 U.S.C. Sections 365 and 120 of International Application No. PCT/EP2006/01151, filed Feb. 9, 2006. This application also claims priority under 35 U.S.C. Section 119 of German Application No. DE 10 2005 067.4, filed Mar. 18, 2005.

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISC

Not Applicable

### BACKGROUND OF THE INVENTION

(1) Field of the Invention

The present invention relates to agents for dyeing keratinic fibers as well as their use and a corresponding method for dyeing hair. The agents comprise at least one dyestuff precursor of a nature-analogous dyestuff selected from the group of the indole or indoline derivatives, at least one oxidation dyestuff precursor of the developer type as well as at least one oxidation dyestuff precursor of the coupler type, and is free of (a) ammonia and (b) additional oxidizing agents.

Nowadays, human hair is treated in a variety of ways with hair cosmetic preparations. They include, for example, the cleaning of hair with shampoos, care and regeneration with rinses and cures as well as bleaching, dyeing and styling the hair with colorants, toners, permanent wave lotions and styling preparations. Among these, agents for changing or nuancing the color of hair play a prominent role.

(2) Description of Related Art, Including Information Disclosed Under 37 C.F.R. §§ 1.97 and 1.98

For temporary colorations, usually colorants or toners are used that comprise “substantives” as the coloring component. These are dye molecules that are directly absorbed onto the hair and do not require any oxidative process to develop the color. These dyes include, for example, Henna that was already known in antiquity for dyeing skin and hair. These colorations are generally sensitive to shampooing, with the result that a variety of unwanted nuance shifts or even a visible “decoloration” can occur.

A further possibility to dye keratinic fibers is by the use of colorants that comprise a combination of the components

A reactive carbonyl compounds, i.e., compounds containing at least one reactive carbonyl group, and component

B compounds selected from (a) CH-acidic compounds, (b) compounds containing primary or secondary amino groups or hydroxyl groups, selected from primary or secondary aromatic amines, nitrogen-containing heterocyclic compounds and aromatic hydroxy compounds, (c) amino acids, (d) oligopeptides synthesized from 2 to 9 amino acids. The above-cited components A and B are generally not dyestuffs themselves, and therefore are not all suitable per se for dyeing keratinic fibers. In combination, they form dyestuffs in a non-oxidative process. However, among the compounds of components B, suitable oxidation dyestuff precursors of the developer type and/or coupler type also find use with or without added oxidizing agents. In this way this dyeing method (in the following called oxo dyeing) can be directly combined with the oxidative dyestuff system. In the following, the components A and B are referred to as the oxo dye precursors). Oxo dyeing is described, for example, in the publications WO-A1-99/18916, WO-A1-00/38638, WO-A1-01/34106 and WO-A1-01/47483.

The use of onium aldehydes and ketones, in particular, 2- and 4-formyl-1-methylquinolinium compounds, which, in combination with compounds containing primary or secondary amino groups or hydroxyl groups, selected from primary or secondary aromatic amines, nitrogen-containing heterocyclic compounds and aromatic hydroxy compounds, and/or CH-acidic compounds are employed for dyeing keratinic fibers, is disclosed in the publications WO-A2-99/18916 and WO-A1-01/47483.

The so-called oxidation dyes are used for long-lasting, intensive colorations with corresponding authentic characteristics. Such dyes usually comprise oxidation dyestuff precursors of the developer type (developer components) and the coupler type (coupler components). Under the influence of oxidizing agents or from atmospheric oxygen, the developer components form the actual colorants among each other or by coupling with one or more coupler components. The oxidation dyes are distinguished by outstanding, long-lasting coloration results.

Finally, another dyeing method has attracted lots of interest. In this method, precursors of the natural hair dyestuff melanin are applied onto the hair; in the course of oxidative processes they then form analogs to natural colorants in the hair. This type of process with 5,6-dihydroxyindoline as the dyestuff precursor was described in EP-B1-530 229. By using, especially repeated use, of agents with 5,6-dihydroxyindoline it is possible to restore the natural hair color to people with gray hair. In this way the coloration can take place with atmospheric oxygen as the sole oxidizing agent, with the result that no recourse is needed to other oxidizing agents.

Preferably, there occurs a gentle dyeing with atmospheric oxygen. The typically employed dyestuff precursors based on indole or indoline are incorporated for this purpose into a cosmetic carrier that preferably has a basic pH. The coloration from this method results in keratinic fibers with a natural black, brown or blond color that particularly in the brown and blond region possesses a slightly red, blue or violet color nuance. If additional oxidizing agents are used in the colorants, then these color shifts appear to a negligible degree or not at all.

Colorants based on nature-analogous dyestuffs address those consumers who wish to give back a natural hair color to their gray hair using a gentle treatment. The abovementioned color shift is particularly undesirable for these consumers. Consequently, for nature-analogous dyes, there is room for improvement in this regard.

Hair dyes based on dyestuff precursors of the indole or the indoline type, which additionally comprise at least one amino acid or an oligopeptide for improving the coloration on gray hair, are known from the publication EP-B1-1 098 627. All the resulting colorations on blond hair possess an unwanted red or blue shade.

Hair dyes are known from the publication EP-B1-613 366, which comprise, in addition to a dyestuff precursor of the indoline type, 0.05 to 5 wt. % of at least one dyestuff precursor of the developer type as well as 0.05 to 5 wt. % of at least one oxidation dyestuff precursor of the coupler type. It was found that the indoline derivatives improve the color properties of the usual oxidation dyes based on developers and couplers.

The publication EP-A2-1 254 650 discloses hair dyes that comprise, in addition to indoline derivatives as the hair dyestuff precursor, at least one selected organic primary amine as the alkalizing agent. Brown and black colorations are supposed to be obtained without red color shifts on using this hair dye. In any case, the results obtained with these dyes are unsatisfactory.

### BRIEF SUMMARY OF THE INVENTION

The present invention pertains to an agent for atmospheric oxygen-induced dyeing of keratinic fibers, especially human hair, comprising a cosmetically acceptable carrier and
(a) at least one derivative of indole and/or indoline as the dyestuff precursor of a nature-analogous dyestuff,
(b) at least one oxidation dyestuff precursor of the developer type,
(c) at least one oxidation dyestuff precursor of the coupler type and
(d) at least one alkalization agent, with the proviso that
   the agent is free of ammonia and
   the agent is free of additional oxidizing agents capable of oxidizing components (a), (b) and (c).

It was surprisingly found that under atmospheric oxidation, dyes based on derivatives of indole or indoline lead to intensive, long-lasting and natural colorations without color shifts, when at least one alkalization agent as well as oxidation dyestuff precursors of the developer type and the coupler type are additionally comprised in this agent and the agents are free of ammonia.

### BRIEF DESCRIPTION OF THE SEVERAL VIEWS OF THE DRAWING(S)

Not Applicable

### DETAILED DESCRIPTION OF THE INVENTION

One aspect of the present invention is an agent for atmospheric oxygen-induced dyeing of keratinic fibers, especially human hair, comprising a cosmetically acceptable carrier and
(a) at least one derivative of indole and/or indoline as the dyestuff precursor of a nature-analogous dyestuff,
(b) at least one oxidation dyestuff precursor of the developer type,
(c) at least one oxidation dyestuff precursor of the coupler type and
(d) at least one alkalization agent, with the proviso that
   the agent is free of ammonia and
   the agent is free of additional oxidizing agents capable of oxidizing components (a), (b) and (c).

According to the invention, keratinic fibers are understood to mean furs, wool, feathers and particularly human hair. Although the inventive dyes are primarily suitable for dyeing keratinic fibers, in principle, nothing prevents their use in other fields of coloration, as long as the technical object on which the invention is based is achieved.

Compounds according to Formula (1a) and/or their physiologically compatible salts with an organic or inorganic acid are preferably comprised in the inventive agents as the indoline derivative of feature (a) of the inventive agent, 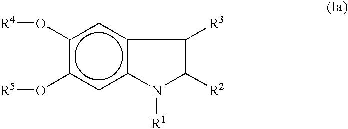
in which
R¹ stands for hydrogen, a C₁-C₄ alkyl group or a C₁-C₄ hydroxyalkyl group,
R² stands for hydrogen or a —COOH group, where the —COOH group may also be present as the salt with a physiologically compatible cation,
R³ stands for hydrogen or a C₁-C₄ alkyl group,
R⁴ stands for hydrogen, a C₁-C₄ alkyl group or a —CO—R⁶ group, in which R⁶ stands for a C₁-C₄alkyl group, and
R⁵ stands for one of the groups cited for R⁴.

Particularly preferred derivatives of indoline are 5,6-dihydroxyindoline, N-methyl-5,6-dihydroxyindoline, N-ethyl-5,6-dihydroxyindoline, N-propyl-5,6-dihydroxyindoline, N-butyl-5,6-dihydroxyindoline, 5,6-dihydroxyindoline-2-carboxylic acid and 6-hydroxyindoline, 6-aminoindoline and 4-aminoindoline.

Within this group, emphasis is placed particularly on N-methyl-5,6-dihydroxyindoline, N-ethyl-5,6-dihydroxyindoline, N-propyl-5,6-dihydroxyindoline, N-butyl-5,6-dihydroxyindoline and, in particular, 5,6-dihydroxyindoline.

Derivatives of 5,6-Dihydroxyindole according to Formula (1b) and/or their physiologically compatible salts with an organic or inorganic acid are also suitable as the precursors of nature-analogous hair dyestuffs according to feature (a), 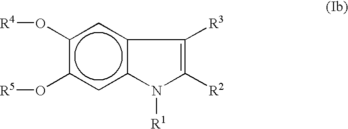
in which
R¹ stands for hydrogen, a C₁-C₄ alkyl group or a C₁-C₄ hydroxyalkyl group,
R² stands for hydrogen or a —COOH group, where the —COOH group may also be present as the salt with a physiologically compatible cation,
R³ stands for hydrogen or a C₁-C₄ alkyl group,
R⁴ stands for hydrogen, a C₁-C₄ alkyl group or a —CO—R⁶ group, in which R⁶ stands for a C₁-C₄alkyl group, and
R⁵ stands for one of the groups cited for R⁴,
and physiologically compatible salts of these compounds with an organic or inorganic acid.

Particularly preferred derivatives of indole are 5,6-dihydroxyindole, N-methyl-5,6-dihydroxyindole, N-ethyl-5,6-dihydroxyindole, N-propyl-5,6-dihydroxyindole, N-butyl-5,6-dihydroxyindole, 5,6-dihydroxyindole-2-carboxylic acid, 6-hydroxyindole, 6-aminoindole and 4-aminoindole.

Within this group, emphasis is given to N-methyl-5,6-dihydroxyindole, N-ethyl-5,6-dihydroxyindole, N-propyl-5,6-dihydroxyindole, N-butyl-5,6-dihydroxyindole and, in particular, 5,6-dihydroxyindole.

The indoline and indole derivatives can be employed in the inventive colorants both as free bases and also in the form of their physiologically compatible salts of inorganic or organic acids, e.g., the hydrochlorides, the sulfates and hydrobromides.

The dyestuff precursors of the nature-analogous dyes are preferably comprised in the inventive agents in amounts of 0.01 to 10 wt. %, particularly 0.1 to 5 wt. %, each based on the weight of the ready for use colorant.

The dyestuff precursors of the oxidation dyes of the developer type of feature (b) of the inventive agent are preferably comprised according to the invention in an amount of 0.01 to 5 wt. %, particularly 0.1 to 3 wt. %, each based on the weight of the ready for use colorant.

According to the invention, it can be preferred to select the inventive developer components from the group formed from p-phenylenediamine derivatives, binuclear developer components, p-amino phenol and its derivatives, pyrimidine derivatives, pyrazole derivatives and pyrazole pyrimidine derivatives and the physiologically compatible salts of these compounds. Inventively preferred developer components are cited below.

According to the invention, particular preference is given to p-phenylenediamine derivatives of the formula (E1) 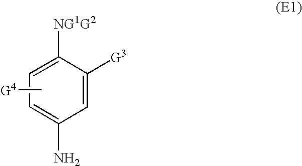
in which
G¹ stands for a hydrogen atom, a C₁- to C₄ alkyl group, a C₁- to C₄ monohydroxyalkyl group, a C₂- to C₄ polyhydroxyalkyl group, a (C₁- to C₄) alkoxy(C₁- to C₄) alkyl group, a 4′-aminophenyl group or a C₁- to C₄ alkyl group that is substituted by a nitrogen-containing group, a phenyl group or a 4′-aminophenyl group;
G² stands for a hydrogen atom, a C₁- to C₄ alkyl group, a C₁- to C₄ monohydroxyalkyl group, a C₂- to C₄ polyhydroxyalkyl group, a (C₁- to C₄) alkoxy(C₁- to C₄) alkyl group or a C₁- to C₄ alkyl group that is substituted by a nitrogen-containing group;
G³ stands for a hydrogen atom, a halogen atom, such as a chlorine, bromine, iodine or fluorine atom, a C₁- to C₄ alkyl group, a C₁- to C₄ monohydroxyalkyl group, a C₂- to C₄ polyhydroxyalkyl group, a (C₁- to C₄) hydroxyalkyl group, a C₁- to C₄ acetylamino alkoxy group, a C₁- to C₄ mesylamino alkoxy group or a C₁- to C₄ carbamoylamino alkoxy group;
G⁴ stands for a hydrogen atom a halogen atom or a C₁- to C₄ alkyl group or when G³ and G⁴ are in the ortho position relative to one another, they can together form a bridging a,ω-alkylenedioxo group, such as, for example, an ethylenedioxy group.

Examples of the C₁- to C₄ alkyl groups specified as substituents in the compounds according to the invention are the methyl, ethyl, propyl, isopropyl and butyl groups. Ethyl and methyl are preferred alkyl groups. Inventively preferred C₁- to C₄ alkoxy groups are a methoxy or an ethoxy group, for example. Furthermore, preferred examples of a C₁- to C₄ hydroxyalkyl group that may be mentioned are a hydroxymethyl, a 2-hydroxyethyl, a 3-hydroxypropyl or a 4-hydroxybutyl group. A 2-hydroxyethyl group is particularly preferred. A particularly preferred C₂- to C₄ polyhydroxyalkyl group is the 1,2-dihydroxyethyl group. According to the invention, examples of halogen atoms are F, Cl or Br atoms, Cl atoms being quite particularly preferred. The other terms used are derived according to the invention from the definitions given here. Examples of nitrogen-containing groups of the formula (E1) are, in particular, the amino groups, C₁- to C₄ monoalkylamino groups, C₁- to C₄ dialkylamino groups, C₁- to C₄ trialkylammonium groups, C₁- to C₄ monohydroxyalkylamino groups, imidazolinium and ammonium.

Particularly preferred p-phenylenediamines of the formula (E1) are chosen from p-phenylenediamine, p-toluenediamine, 2-chloro-p-phenylenediamine, 2,3-dimethyl-p-phenylenediamine, 2,6-dimethyl-p-phenylenediamine, 2,6-diethyl-p-phenylenediamine, 2,5-dimethyl-p-phenylenediamine, N,N-dimethyl-p-phenylenediamine, N,N-diethyl-p-phenylenediamine, N,N-dipropyl-p-phenylenediamine, 4-amino-3-methyl-(N,N-diethyl)aniline, N,N-bis-(β-hydroxyethyl)-p-phenylenediamine, 2-hydroxymethyl-p-phenylenediamine, 4-N,N-bis(β-hydroxyethyl)-amino-2-chloroaniline, 2-(β-hydroxyethyl)-p-phenylenediamine, 2-(α,β-dihydroxyethyl)-p-phenylenediamine, 2-fluoro-p-phenylenediamine, 2-isopropyl-p-phenylenediamine, N-(β-hydroxypropyl)-p-phenylenediamine, 2-hydroxymethyl-p-phenylenediamine, N,N-dimethyl-3-methyl-p-phenylenediamine, N,N-(ethyl-β-hydroxyethyl)-p-phenylenediamine, N-(β,γ-dihydroxypropyl)-p-phenylenediamine, N-(4′-aminophenyl)-p-phenylenediamine, N-phenyl-p-phenylenediamine, 2-(β-hydroxyethyloxy)-p-phenylenediamine, 2-(β-acetylaminoethyloxy)-p-phenylenediamine, N-(β-methoxyethyl)-p-phenylenediamine and 5,8-diaminobenzo-1,4-dioxane, and their physiologically compatible salts.

According to the invention, quite particularly preferred p-phenylenediamine derivatives of Formula (E1) are p-phenylenediamine, p-toluenediamine, 2-(β-hydroxyethyl)-p-phenylenediamine, 2-(α,β-dihydroxyethyl)-p-phenylenediamine and N,N-bis-(β-hydroxyethyl)-p-phenylenediamine.

According to the invention, it may also be preferred to use compounds as the developer component, which comprise at least two aromatic nuclei that are substituted by amino and/or hydroxyl groups.

Among the binuclear developer components that can be used in the colorant compositions according to the invention, mention may be made, in particular, of the compounds which conform to the following formula (E2), together with their physiologically compatible salts: 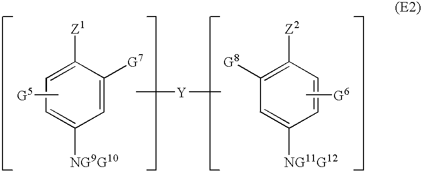
wherein:
Z¹ and Z², independently of one another, stand for a hydroxyl or NH₂ group, which is optionally substituted by a C₁- to C₄ alkyl group, by a C₁- to C₄ hydroxyalkyl group and/or by a bridge Y or which is optionally part of a bridging ring system,
the bridge Y stands for an alkylene group containing 1 to 14 carbon atoms, such as, for example, a linear or branched alkylene chain or an alkylene ring, which can be interrupted or terminated by one or more nitrogen-containing groups and/or one or more heteroatoms, such as oxygen, sulfur or nitrogen atoms and may possibly be substituted by one or more hydroxyl or C₁- to C₈ alkoxy groups, or is a direct bond,
G⁵ and G⁶, independently of one another, stand for a hydrogen or halogen atom, a C₁- to C₄ alkyl group, a C₁- to C₄ monohydroxyalkyl group, a C₂- to C₄ polyhydroxyalkyl group, a C₁- to C₄ aminoalkyl group or a direct bond to the bridge Y,
G⁷, G⁸, G⁹, G¹⁰, G¹¹ and G¹², independently of one another, stand for a hydrogen atom, a direct bond to the bridge Y or a C₁- to C₄ alkyl group, with the proviso that the compounds of Formula (E2) comprise only one bridge Y per molecule.

According to the invention, the substituents in formula (E2) are defined analogously to the above statements.

Preferred binuclear developer components of the formula (E2) are, in particular: N,N′-bis-(β-hydroxyethyl)-N,N′-bis-(4′-aminophenyl)-1,3-diamino-propane-2-ol, N,N′-bis-(β-hydroxyethyl)-N,N′-bis-(4′-aminophenyl)-ethylenediamine, N,N′-bis-(4-aminophenyl)-tetramethylenediamine, N,N′-bis-(β-hydroxyethyl)-N,N′-bis-(4-aminophenyl)-tetramethylenediamine, N,N′-bis-(4-methylaminophenyl)-tetramethylenediamine, N,N′-diethyl-N,N′-bis-4′-amino-3′-methylphenylethylenediamine, bis-(2-hydroxy-5-aminophenyl)-methane, N,N′-bis-(4′-aminophenyl)-1,4-diazacycloheptane, N,N′-bis-(2-hydroxy-5-aminobenzyl)-piperazine, N-(4′-aminophenyl)-p-phenylenediamine and 1,10-bis-(2′,5′-diaminophenyl)-1,4,7,10-tetraoxadecane and their physiologically compatible salts.

Quite particularly preferred binuclear developer components of the formula (E2) are N,N′-bis(β-hydroxyethyl)-N,N′-bis(4-aminophenyl)-1,3-diaminopropane-2-ol, bis(2-hydroxy-5-aminophenyl)methane, 1,3-bis(2,5-diaminophenoxy)propane-2-ol, N,N′-bis(4-aminophenyl)-1,4-diazacycloheptane and 1,10-bis(2,5-diaminophenyl)-1,4,7,10-tetraoxadecane or one of their physiologically compatible salts.

Moreover, according to the invention, it may be preferred to use a p-amino phenol or a p-amino phenol derivative or one of the physiologically compatible salts of the cited compounds as the developer component. p-Amino phenol derivates of the Formula (E3) are particularly preferred. 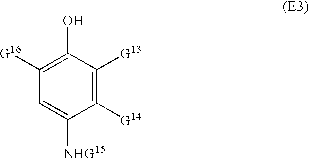
wherein:
G¹³ stands for a hydrogen atom, a halogen atom, a C₁- to C₄ alkyl group, a C₁- to C₄ monohydroxyalkyl group, a C₂- to C₄ polyhydroxyalkyl group, a (C₁- to C₄) alkoxy-(C₁- to C₄) alkyl group, a C₁- to C₄ aminoalkyl group, a hydroxy-(C₁- to C₄) alkylamino group, a C₁- to C₄ hydroxyalkoxy group, a C₁- to C₄ hydroxyalkyl-(C₁- to C₄-) aminoalkyl group or a (di-C₁- to C₄ alkylamino)-(C₁- to C₄) alkyl group, and
G¹⁴ stands for a hydrogen or halogen atom, a C₁- to C₄ alkyl group, a C₁- to C₄ monohydroxyalkyl group, a C₂- to C₄ polyhydroxyalkyl group, a (C₁- to C₄) alkoxy-(C₁- to C₄) alkyl group, C₁- to C₄ aminoalkyl group or a C₁- to C₄ cyanoalkyl group,
G¹⁵ stands for hydrogen, a C₁- to C₄ alkyl group, a C₁- to C₄ monohydroxyalkyl group, a C₂- to C₄ polyhydroxyalkyl group, a phenyl group or a benzyl group, and
G¹⁶ stands for hydrogen or a halogen atom.

According to the invention, the substituents in formula (E3) are defined analogously to the above statements.

Preferred p-amino phenols of the Formula (E3) are especially p-amino phenol, N-methyl-p-amino phenol, 4-amino-3-methyl-phenol, 4-amino-3-fluorophenol, 2-hydroxymethylamino-4-amino phenol, 4-amino-3-hydroxymethylphenol, 4-amino-2-(β-hydroxyethoxy)phenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)phenol, 4-amino-2-(α,β-dihydroxyethyl)phenol, 4-amino-2-fluorophenol, 4-amino-2-chlorophenol, 4-amino-2,6-dichlorophenol, 4-amino-2-(diethylaminomethyl)phenol together with their physiologically compatible salts.

Quite particularly preferred compounds of the Formula (E3) are p-amino phenol, 4-amino-3-methylphenol, 4-amino-2-aminomethylphenol, 4-amino-2-(α,β-dihydroxyethyl)phenol and 4-amino-2-(diethylaminomethyl)phenol.

Furthermore, the developer component can be selected from o-amino phenol and its derivatives, such as, for example, 2-amino-4-methylphenol, 2-amino-5-methylphenol or 2-amino-4-chlorophenol.

In addition, the developer component can be chosen from heterocyclic developer components, such as, for example, the pyridine, pyrimidine, pyrazole, pyrazole-pyrimidine derivatives and their physiologically compatible salts.

Preferred pyridine derivatives are, in particular, the compounds which are described in the patents GB 1 026 978 and GB 1,153,196, such as 2,5-diaminopyridine, 2-(4-methoxyphenyl)amino-3-amino pyridine, 2,3-diamino-6-methoxypyridine, 2-(β-methoxyethyl)amino-3-amino-6-methoxypyridine and 3,4-diamino pyridine.

According to the invention, preferred pyrimidine derivatives are selected from compounds of Formula (E4), 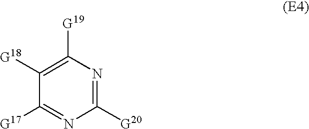
in which
G¹⁷, G¹⁸ and G¹⁹ independently of one another stands for a hydrogen atom, a hydroxyl group, a (C₁-C₆) alkoxy group or an amino group and
G²⁰ stands for a hydroxyl group or a —NG²¹G²² group, in which G²¹ and G²² independently of one another stand for a hydrogen atom, a (C₁-C₆) alkyl group, a (C₁-C₆) hydroxyalkyl group,

with the proviso that at most two of the groups G¹⁷, G¹⁸, G¹⁹ and G²⁰ mean a hydroxyl group and at most two of the groups G¹⁷, G¹⁸ and G¹⁹ stand for a hydrogen atom.

Particularly preferred pyrimidine derivatives are, in particular, the compounds which are described in the German patent DE 2 359 399, the Japanese laid-open specification JP 02019576 A2 or in the laid-open specification WO 96/15765, such as 2,4,5,6-tetramino pyrimidine, 4-hydroxy-2,5,6-triamino pyrimidine, 2-hydroxy-4,5,6-triamino pyrimidine, 2-dimethylamino-4,5,6-triamino pyrimidine, 2,4-dihydroxy-5,6-diamino pyrimidine and 2,5,6-triamino pyrimidine.

According to the invention, preferred pyrazole derivatives are selected from compounds of Formula (E5), 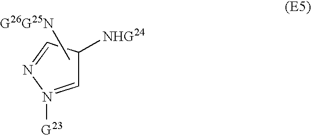
in which
G²³, G²⁴, G²⁵ independently of one another, stand for a hydrogen atom, a C₁ to C₆ alkyl group, a C₂ to C₆ monohydroxyalkyl group, a C₂ to C₆ polyhydroxyalkyl group, an optionally substituted aryl group or an optionally substituted aryl C₁ to C₆ alkyl group and
G²⁶ stands for hydrogen atom, a C₁ to C₆ alkyl group, a C₂ to C₆ m monohydroxyalkyl group or a C₂ to C₆ polyhydroxyalkyl group.

Preferred pyrazole derivatives are, in particular, the compounds which are described in the patents DE 3,843,892, DE 4,133,957 and patent applications WO 94/08969, WO 94/08970, EP 740,931 and DE 195 43 988, such as 4,5-diamino-1-methylpyrazole, 4,5-diamino-1-(β-hydroxyethyl)-pyrazole, 3,4-diamino pyrazole, 4,5-diamino-1-(4′-chlorobenzyl)-pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert.-butyl-1-methylpyrazole, 4,5-diamino-1-tert.-butyl-3-methylpyrazole, 4,5-diamino-1-(β-hydroxyethyl)-3-methylpyrazole, 4,5-diamino-1-ethyl-3-methylpyrazole, 4,5-diamino-1-ethyl-3-(4′-methoxyphenyl)-pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(β-aminoethyl)-amino-1,3-dimethylpyrazole.

Preferred pyrazolo pyrimidine derivatives are, in particular, the derivatives of the pyrazolo[1,5-a]pyrimidine of the following formula (E6) and its tautomeric forms, provided there is a tautomeric equilibrium: 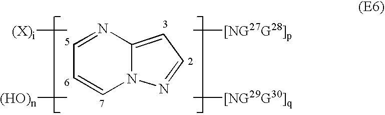
in which

G²⁷, G²⁸, G²⁹ and G³⁰ independently of one another stand for a hydrogen atom, a C₁- to C₄ alkyl group, an aryl group, a C₁- to C₄ hydroxyalkyl group, a C₂- to C₄ polyhydroxyalkyl group a (C₁- to C₄) alkoxy-(C₁- to C₄) alkyl group, a C₁- to C₄-aminoalkyl group, optionally protected by an acetyl-ureido or a sulfonyl group, a (C₁- to C₄) alkylamino (C₁- to C₄) alkyl group, a di-[(C₁- to C₄)-alkyl]-(C₁- to C₄) aminoalkyl group, wherein the dialkyl groups optionally form a carbocycle or a heterocycle with 5 or 6 chain members, a C₁- to C₄ hydroxyalkyl or a di-(C₁- to C₄)-[hydroxyalkyl]-(C₁- to C₄) aminoalkyl group,

the X groups independently of one another stand for a hydrogen atom, a C₁-bis C₄ alkyl group, an aryl group, a C₁- to C₄ hydroxyalkyl group, a C₂- to C₄ polyhydroxyalkyl group, a C₁- to C₄ aminoalkyl group, a (C₁- to C₄) alkylamino-(C₁- to C₄) alkyl group, a di-[(C₁- to C₄)-alkyl]-(C₁- to C₄) aminoalkyl group, wherein the dialkyl groups optionally form a carbocycle or a heterocycle with 5 or 6 chain members, a C₁- to C₄ hydroxyalkyl or a di-(C₁- to C₄ hydroxyalkyl)-(C₁- to C₄) aminoalkyl group, an amino group, a C₁- to C₄ alkyl- or a di-(C₁- to C₄ hydroxyalkyl)amino group, a halogen atom, a carboxylic acid group or a sulfonic acid group,
i has the value 0, 1, 2 or 3,
p has the value 0 or 1,
q has the value 0 or 1 and
n has the value 0 or 1,
   with the proviso that
the sum of p+q is not equal to 0,
if p+q is equal to 2, then n has the value 0, and the groups NG²⁷G²⁸ and NG²⁹G³⁰ occupy the positions (2,3); (5,6); (6,7); (3,5) or (3,7);
if p+q is equal to 1, then n has the value 1, and the groups NG²⁷G²⁸ (or NG²⁹G³⁰) and the group OH occupy the positions (2,3); (5,6); (6,7); (3,5) or (3,7);

According to the invention, the substituents in formula (E6) are defined analogously to the above statements.

If the pyrazolo[1,5-a]pyrimidine of the above formula (E6) comprises a hydroxyl group in one of the positions 2, 5 or 7 of the ring system, there is a tautomeric equilibrium, which is illustrated, for example, in the following scheme:

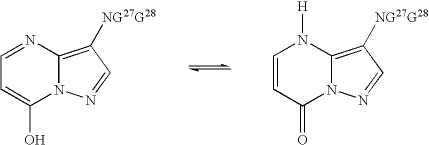

Among the pyrazolo[1,5-a]pyrimidines of the above formula (E6), mention may be made in particular, of:
Pyrazolo[1,5-a]pyrimidine-3,7-diamine;
2,5-Dimethyl-pyrazolo[1,5-a]pyrimidine-3,7-diamine;
Pyrazolo[1,5-a]pyrimidine-3,5-diamine;
2,7-Dimethyl-pyrazolo[1,5-a]pyrimidine-3,5-diamine;
3-Aminopyrazolo[1,5-a]pyrimidine-7-ol;
3-Aminopyrazolo[1,5-a]pyrimidine-5-ol;
2-(3-Aminopyrazolo[1,5-a]pyrimidin-7-ylamino)ethanol;
2-(7-Aminopyrazolo[1,5-a]pyrimidin-3-ylamino)ethanol;
2-[(3-Aminopyrazolo[1,5-a]pyrimidin-7-yl)-(2-hydroxyethyl)amino]ethanol;
2-[(7-Aminopyrazolo[1,5-a]pyrimidin-3-yl)-(2-hydroxyethyl)amino]ethanol;
5,6-Dimethylpyrazolo[1,5-a]pyrimidine-3,7-diamine;
2,6-Dimethylpyrazolo[1,5-a]pyrimidine-3,7-diamine;
3-Amino-7-dimethylamino-2,5-dimethylpyrazolo[1,5-a]pyrimidine;

and their physiologically compatible salts and their tautomeric forms if a tautomeric equilibrium is present.

The pyrazolo[1,5-a]pyrimidines of the above formula (E6) can be prepared as described in the literature by cyclization starting from an amino pyrazole or from hydrazine.

m-Phenylenediamine derivatives, naphthols, resorcinol and resorcinol derivatives, pyrazolones and m-amino phenol derivatives are generally used as the coupling components. Particularly suitable coupling substances are 1-naphthol, 1,5-dihydroxynaphthalene, 2,7-dihydroxynaphthalene, and 1,7-dihydroxynaphthalene, 5-amino-2-methylphenol, m-amino phenol, resorcinol, resorcinol monomethyl ether, m-phenylenediamine, 1-phenyl-3-methylpyrazolone-5, 2,4-dichloro-3-aminophenol, 1,3-bis(2′,4′-diamino phenoxy)propane, 2-chlororesorcinol, 4-chlororesorcinol, 2-chloro-6-methyl-3-amino phenol, 2-amino-3-hydroxypyridine, 2-methylresorcinol, 5-methylresorcinol and 2-methyl-4-chloro-5-aminophenol.

Inventively preferred coupler components (c) are selected from at least one member of the group of compounds formed by
m-amino phenol and derivatives thereof such as preferably 5-amino-2-methylphenol, N-cyclopentyl-3-amino phenol, 3-amino-2-chloro-6-methylphenol, 2-hydroxy-4-amino phenoxyethanol, 2,6-dimethyl-3-amino phenol, 3-trifluoroacetylamino-2-chloro-6-methylphenol, 5-amino-4-chloro-2-methylphenol, 5-amino-4-methoxy-2-methylphenol, 5-(2′-hydroxyethyl)amino-2-methylphenol, 3-(diethylamino)phenol, N-cyclopentyl-3-amino phenol, 1,3-dihydroxy-5-(methylamino)benzene, 3-(ethylamino)-4-methylphenol and 2,4-dichloro-3-amino phenol,
o-amino phenol and derivatives thereof,
m-phenylenediamine and derivatives thereof such as preferably 2,4-diamino phenoxyethanol, 1,3-bis-(2′,4′-diamino phenoxy)propane, 2-amino-1-methoxy-4-(2-hydroxyethylamino)benzene, 1,3-bis-(2′,4′-diaminophenyl)propane, 2,6-bis-(2′-hydroxyethylamino)-1-methylbenzene and 1-amino-3-bis-(2′-hydroxyethyl)amino benzene, 2-({3-[(2-hydroxyethyl)amino]2-methoxy-5-methylphenyl}amino)ethanol, 3-amino-4-(2-methoxyethoxy)-5-methylphenylamine, 2-({3-[(2-hydroxyethyl)amino]-4-methoxy-5-methylphenyl}amino)ethanol and 2-[(3-morpholin-4-ylphenyl)amino]ethanol,
o-phenylenediamine and derivatives thereof such as preferably 3,4-diaminobenzoic acid and 2,3-diamino-1-methylbenzene,
Di- or trihydroxybenzene derivatives, such as preferably resorcinol and derivatives (resorcinol, resorcinol monomethyl ether, 2-methylresorcinol, 5-methylresorcinol, 2,5-dimethylresorcinol, 2-chlororesorcinol, 4-chlororesorcinol), pyrogallol and 1,2,4-trihydroxybenzene,
Pyridine derivatives such as preferably 2,6-dihydroxypyridine, 2-amino-3-hydroxypyridine, 2-amino-5-chloro-3-hydroxypyridine, 3-amino-2-methylamino-6-methoxypyridine, 2,6-dihydroxy-3,4-dimethylpyridine, 2,6-dihydroxy-4-methylpyridine, 2,6-diaminopyridine, 2,3-diamino-6-methoxypyridine and 3,5-diamino-2,6-dimethoxypyridine,
Naphthalene derivatives, such as preferably mono- or dihydroxynaphthalene derivatives, such as, for example, 1-naphthol, 2-methyl-1-naphthol, 2-hydroxymethyl-1-naphthol, 2-hydroxyethyl-1-naphthol, 1,5-dihydroxynaphthalene, 1,6-dihydroxynaphthalene, 1,7-dihydroxynaphthalene, 1,8-dihydroxynaphthalene, 2,7-dihydroxynaphthalene, 1,3-dihydroxynaphthalene and 2,3-dihydroxynaphthalene,
Morpholine derivatives such as preferably 6-hydroxybenzomorpholine and 6-amino-benzomorpholine,
Quinoxaline derivatives such as preferably 6-methyl-1,2,3,4-tetrahydroquinoxaline,
Pyrazolone derivatives such as preferably 1-phenyl-3-methylpyrazol-5-one and
Methylenedioxybenzene derivatives such as preferably 1-hydroxy-3,4-methylenedioxybenzene, 1-amino-3,4-methylenedioxybenzene and 1-(2′-hydroxyethyl)-amino-3,4-methylenedioxybenzene.

In the context of the invention, it is preferred to combine the dyestuff precursors of nature-analogous dyestuffs of the indole or indoline type with at least one of the following combinations of developer and coupler components, as is shown in Table 1.

TABLE 1
Preferred Combinations of Developer and Coupler Components.

| Combination | Developer component | Coupler component |
|---|---|---|
| K1 | p-Phenylenediamine derivative, | Resorcinol and |
| | preferably according to Formula | derivatives thereof |
| | (EI) | |
| K2 | Pyrazole derivative, preferably | Resorcinol and |
| | according to Formula (E5) | derivatives thereof |
| K3 | Pyrimidine derivative, preferably | Resorcinol and |
| | according to Formula (E4) | derivatives thereof |
| K4 | Binuclear developer preferably | Resorcinol and |
| | according to Formula (E2) | derivatives thereof |
| K5 | p-Amino phenol derivative | m-Phenylenediamine |
| | preferably according to Formula | and derivatives |
| | (E3) | thereof |
| K6 | Binuclear developer preferably | m-Phenylenediamine |
| | according to Formula (E2) | and derivatives thereof |
| K7 | p-Phenylenediamine derivative, | Pyridine derivative |
| | preferably according to Formula | |
| | (EI) | |
| K8 | Binuclear developer preferably | Pyridine derivative |
| | according to Formula (E2) | |
| K9 | p-Amino phenol derivative | Pyridine derivative |
| | preferably according to | |
| | Formula (E3) | |
| K10 | Binuclear developer preferably | m-Amino phenol and |
| | according to Formula (E2) | derivatives thereof |
| K11 | Pyrazole derivative, preferably | m-Amino phenol and |
| | according to Formula (E5) | derivatives thereof |
| K12 | p-Phenylenediamine derivative, | 5-(2-Hydroxyethyl- |
| | preferably according to Formula | amino)-2-methyl-phenol |
| | (EI) | |

The respective developer and coupler components in Table 1 are preferably selected from the preferred individual representatives and/or their physiologically compatible salts cited in the context of each of the classes of compounds. It is quite particularly preferred that the inventive agents comprise at least one of the following combinations of features for the features (b) and (c).

Particularly preferred combinations for K1 from Table 1 are:
p-Toluenediamine and resorcinol,
2-(β-Hydroxyethyl)-p-phenylenediamine and resorcinol,
p-Toluenediamine and 2-methylresorcinol,
2-(β-Hydroxyethyl)-p-phenylenediamine and 2-methylresorcinol,
p-Toluenediamine and 4-chlororesorcinol,
2-(β-Hydroxyethyl)-p-phenylenediamine and 4-chlororesorcinol,
2-(β-Hydroxyethyl)-p-phenylenediamine, 2-methylresorcinol and 2-amino-3-hydroxypyridine, and
2-(β-Hydroxyethyl)-p-phenylenediamine, 2-methylresorcinol and 1,5-dihydroxynaphthalene.

Particularly preferred combinations for K2 from Table 1 are:
4,5-Diamino-1-(2-hydroxyethyl)pyrazole and resorcinol,
4,5-Diamino-1-(2-hydroxyethyl)pyrazole and 2-methylresorcinol and
4,5-Diamino-1-(2-hydroxyethyl)pyrazole and 4-chlororesorcinol.

Particularly preferred combinations for K3 from Table 1 are:
2,4,5,6-Tetraminopyrimidine and resorcinol,
2,4,5,6-Tetraminopyrimidine and 2-methylresorcinol,
2,4,5,6-Tetraminopyrimidine and 4-chlororesorcinol,
4-Hydroxy-2,5,6-triaminopyrimidine and resorcinol,
4-Hydroxy-2,5,6-triaminopyrimidine and 2-methylresorcinol,
4-Hydroxy-2,5,6-triaminopyrimidine and 4-chlororesorcinol and
2,4,5,6-Tetraminopyrimidine, 2-methylresorcinol and 1,5-dihydroxynaphthalene.

Particularly preferred combinations for K4 from Table 1 are:
Bis-(2-hydroxy-5-amino-phenyl)methane and resorcinol,
Bis-(2-hydroxy-5-amino-phenyl)methane and 2-methylresorcinol and
Bis-(2-hydroxy-5-amino-phenyl)methane and 4-chlororesorcinol.

Particularly preferred combinations for K5 from Table 1 are:
p-Amino phenol and 2,4-diaminophenoxyethanol,
p-Amino phenol and 2-amino-1-methoxy-4-(2-hydroxyethylamino)benzene,
p-Amino phenol and 2,6-bis-(2′-hydroxyethylamino)-1-methylbenzene
4-Amino-3-methylphenol and 2,4-diaminophenoxyethanol,
4-Amino-3-methylphenol and 2-amino-1-methoxy-4-(2-hydroxyethylamino)benzene and
4-Amino-3-methylphenol and 2,6-bis-(2′-hydroxyethylamino)-1-methylbenzene.

Particularly preferred combinations for K6 from Table 1 are:
Bis-(2-hydroxy-5-aminophenyl)-methane and 2,4-diaminophenoxyethanol,
Bis-(2-hydroxy-5-aminophenyl)-methane and 2-amino-1-methoxy-4-(2-hydroxyethylamino)-benzene and
Bis-(2-hydroxy-5-aminophenyl)-methane and 2,6-bis-(2′-hydroxyethylamino)-1-methyl benzene.

Particularly preferred combinations for K7 from Table 1 are:
p-Toluenediamine and 2-amino-3-hydroxypyridine,
2-(β-Hydroxyethyl)-p-phenylenediamine and 2-amino-3-hydroxypyridine,
p-Toluenediamine and 2,6-dihydroxy-3,4-dimethylpyridine,
2-(β-Hydroxyethyl)-p-phenylenediamine and 2,6-dihydroxy-3,4-dimethylpyridine and
2-(β-Hydroxyethyl)-p-phenylenediamine, 2-amino-3-hydroxypyridine and 2-methylresorcinol.

Particularly preferred combinations for K8 from Table 1 are:
Bis-(2-hydroxy-5-aminophenyl)-methane and 2-amino-3-hydroxypyridine,
Bis-(2-hydroxy-5-aminophenyl)-methane and 3-amino-2-methylamino-6-methoxypyridine,
Bis-(2-hydroxy-5-aminophenyl)-methane and 2,6-dihydroxy-3,4-dimethylpyridine and
Bis-(2-hydroxy-5-aminophenyl)-methane and 3,5-diamino-2,6-dimethoxypyridine.

Particularly preferred combinations for K9 from Table 1 are:
p-Amino phenol and 2-amino-3-hydroxypyridine,
4-Amino-3-methylphenol and 2-amino-3-hydroxypyridine,
p-Amino phenol and 3-amino-2-methylamino-6-methoxypyridine,
4-Amino-3-methylphenol and 3-amino-2-methylamino-6-methoxypyridine,
p-Amino phenol and 2,6-dihydroxy-3,4-dimethylpyridine,
4-Amino-3-methylphenol and 2,6-dihydroxy-3,4-dimethylpyridine,
p-Amino phenol and 3,5-diamino-2,6-dimethoxypyridine and
4-Amino-3-methylphenol and 3,5-diamino-2,6-dimethoxypyridine.

The combination of
i) a p-phenylenediamine, preferably selected from compounds of Formula (E1),
ii) a pyridine derivative and
iii) resorcinol or a derivative thereof,
in particular, the combination 2-(β-hydroxyethyl)-p-phenylenediamine, 2-amino-3-hydroxypyridine and 2-methylresorcinol, is quite particularly preferably comprised in the inventive agents.

Moreover, when adding resorcinol and its derivatives as the coupler component, particularly 2-methylresorcinol, it is particularly preferred to additionally incorporate a naphthalene derivative, particularly at least one of the above cited representatives, particularly preferably 1,5-dihydroxynaphthalene, as an additional coupler component to further improve the technical effect.

For all the previously cited preferred combinations, the physiologically compatible salts of the correspondingly enumerated compounds can also be used.

The dyestuff precursors of the oxidation dyes of the coupler type of feature (c) of the inventive agent, also designated below as the coupler components, are preferably comprised according to the invention in an amount of 0.01 to 5 wt. %, particularly 0.1 to 3 wt. %, each based on the weight of the ready for use colorant.

The dyestuff precursors of the nature-analogous dyestuffs (a) and the developer components (b) are preferably comprised in the inventive agents in a molar ratio of 10 to 1 to 1 to 2, particularly preferably in a molar ratio of 8 to 1 to 2 to 1, quite particularly preferably in a molar ratio of 6 to 1 to 3 to 1.

The coupler components (c) and the developer components (b) are preferably comprised in the inventive agents in a molar ratio of 8 to 1 to 1 to 2, particularly preferably in a molar ratio of 6 to 1 to 2 to 1, quite particularly preferably in a molar ratio of 2.5 to 1 to 4.5 to 1.

The dyestuff precursors of the dyestuffs (a) and the coupler components (c) are preferably comprised in the inventive agents in a molar ratio of 2 to 1 to 1 to 2, particularly preferably in a molar ratio of 1.5 to 1 to 1 to 1.5, quite particularly preferably in a molar ratio of 1.5 to 1 to 1 to 1.

The ready-for-use hair coloration preparation according to the invention should preferably have a pH in the range 6 to 12. The hair dye is particularly preferably applied in an alkaline milieu. The inventively usable alkalization agents (d) are preferably selected from the group formed by the basic amino acids, alkali metal hydroxides, alkanolamines, alkali metal carbonates, alkali metal hydrogen carbonates, alkali metal metasilicates, alkali metal phosphates and alkali metal hydrogen phosphates. Lithium, sodium, potassium, particularly sodium or potassium, are preferred alkali metal ions.

In the context of the invention, the basic amino acids that can be employed as the inventive alkalization agent are preferably selected from the group formed by L-arginine, D-arginine, D,L-arginine, L-histidine, D-histidine, D,L-histidine, L-lysine, D-lysine, D,L-lysine, particularly preferably L-arginine, D-arginine, D,L-arginine.

The alkali metal hydroxides that can be employed as the inventive alkalization agent are preferably selected from the group formed by sodium hydroxide and potassium hydroxide.

The alkanolamines that can be employed as the inventive alkalization agent are preferably selected from primary amines containing a C₂-C₆ alkyl parent substance that carries at least one hydroxyl group. Particularly preferred alkanolamines are selected from the group formed by 2-aminoethan-1-ol (monoethanolamine), 3-aminopropan-1-ol, 4-aminobutan-1-ol, 5-aminopentan-1-ol, 1-aminopropan-2-ol, 1-aminobutan-2-ol, 1-aminopentan-2-ol, 1-aminopentan-3-ol, 1-aminopentan-4-ol, 3-amino-2-methylpropan-1-ol, 1-amino-2-methylpropan-2-ol, 3-aminopropane-1,2-diol, 2-amino-2-methylpropane-1,3-diol. Inventively quite particularly preferred alkanolamines are selected from the group 2-aminoethan-1-ol, 2-amino-2-methylpropan-1-ol and 2-amino-2-methylpropane-1,3-diol.

Preferably, an otherwise usual carrier for agents for dyeing human hair is understood as the cosmetically acceptable carrier. With regard to the features that are essential to the invention, the inventive dyes can be formulated with correspondingly known dyes or comprise typical ingredients for them. Examples of further suitable and inventively preferred ingredients are given below.

The inventive agents preferably comprise the inventive components in a suitable aqueous, alcoholic or aqueous alcoholic carrier. For the purposes of dyeing the hair, such carriers are, for example, creams, emulsions, gels and also surfactant-containing foaming solutions, such as, for example, shampoos, foam aerosols or other preparations that are suitable for use on the hair. However, it is also conceivable to integrate the dyestuff precursors into a powdered or tablet-shaped formulation.

For the purposes of the present invention, aqueous-alcoholic solutions are understood as meaning aqueous solutions comprising 3 to 70% by weight of a C₁-C₄-alcohol, in particular, ethanol or isopropanol. The compositions according to the invention can additionally comprise further organic solvents, such as, for example, methoxybutanol, benzyl alcohol, ethyl diglycol or 1,2-propylene glycol. Preference here is given to all water-soluble organic solvents.

A general composition is presented below, with the proviso that the inventive substance ratio of compound A to compound B is respected.

In addition to the inventive compounds, the dyeing compositions according to the invention in a further embodiment of the present invention can comprise one or a plurality of substantive dyes for nuancing. Substantive dyestuffs are usually nitrophenylenediamines, nitroamino phenols, azo dyes, anthraquinones or indophenols. Preferred substantive dyestuffs are the compounds known under the international designations or trade names HC Yellow 2, HC Yellow 4, HC Yellow 5, HC Yellow 6, HC Yellow 12, Acid Yellow 1, Acid Yellow 10, Acid Yellow 23, Acid Yellow 36, HC Orange 1, Disperse Orange 3, Acid Orange 7, HC Red 1, HC Red 3, HC Red 10, HC Red 11, HC Red 13, Acid Red 33, Acid Red 52, HC Red BN, Pigment Red 57:1, HC Blue 2, HC Blue 12, Disperse Blue 3, Acid Blue 7, Acid Green 50, HC Violet 1, Disperse Violet 1, Disperse Violet 4, Acid Violet 43, Disperse Black 9, Acid Black 1, and Acid Black 52 as well as 1,4-diamino-2-nitrobenzol, 2-amino-4-nitrophenol, 1,4-bis-(β-hydroxyethyl)-amino-2-nitrobenzene, 3-nitro-4-(β-hydroxyethyl)-amino phenol, 2-(2′-hydroxyethyl)amino-4,6-dinitrophenol, 1-(2′-hydroxyethyl)amino-4-methyl-2-nitrobenzene, 1-amino-4-(2′-hydroxyethyl)-amino-5-chloro-2-nitrobenzene, 4-amino-3-nitrophenol, 1-(2′-ureidoethyl)amino-4-nitrobenzene, 4-amino-2-nitrodiphenylamine-2′-carboxylic acid, 6-nitro-1,2,3,4-tetrahydroquinoxaline, 2-hydroxy-1,4-naphthoquinone, picramic acid and its salts, 2-amino-6-chloro-4-nitrophenol, 4-ethylamino-3-nitrobenzoic acid and 2-chloro-6-ethylamino-1-hydroxy-4-nitrobenzene.

In addition, the inventive agents can comprise a cationic substantive dyestuff. Particular preference is given here to
(a) cationic triphenylmethane dyes, such as, for example, Basic Blue 7, Basic Blue 26, Basic Violet 2 and Basic Violet 14,
(b) aromatic systems which are substituted by a quaternary nitrogen group, such as, for example, Basic Yellow 57, Basic Red 76, Basic Blue 99, Basic Brown 16 and Basic Brown 17, and
(c) substantive dyes, which comprise a heterocycle that has at least one quaternary nitrogen atom, as are specified, for example, in EP-A2-998 908 in the claims 6 to 11, which is explicitly incorporated here by reference.

Preferred cationic substantive dyestuffs of group (c) are, in particular, the following compounds:

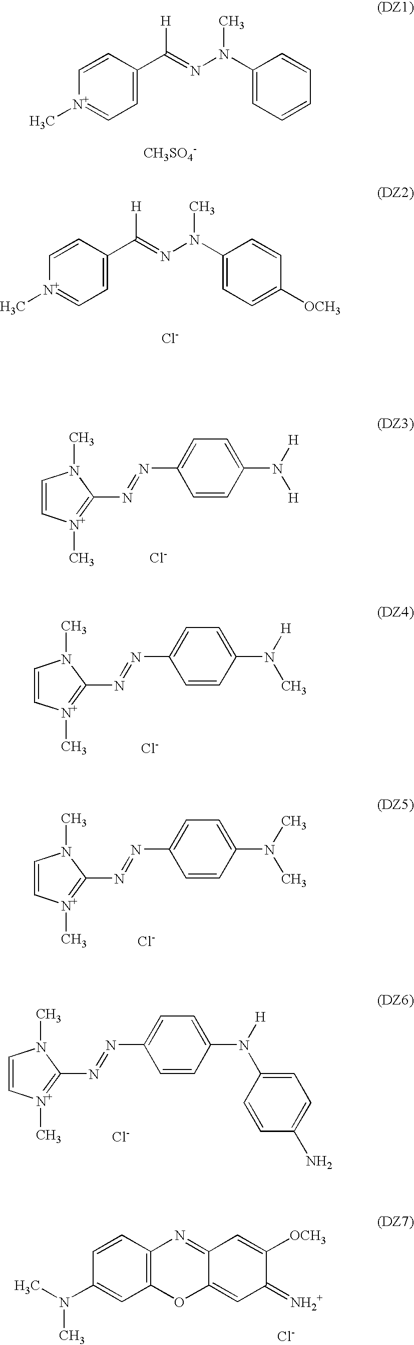

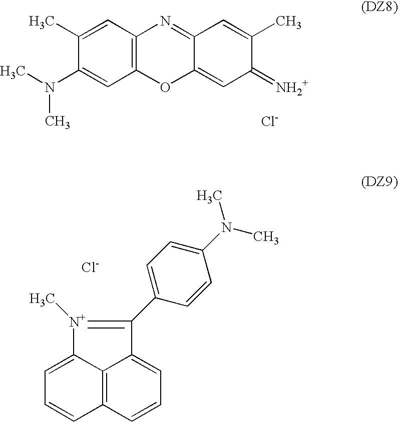

The compounds of the formulas (DZ1), (DZ3) and (DZ5), which are also known under the names Basic Yellow 87, Basic Orange 31 and Basic Red 51, are quite particularly preferred cationic substantive dyestuffs of group (c).

The cationic substantive dyes that are commercialized under the trade name Arianor® are likewise quite particularly preferred cationic substantive dyestuffs according to the invention.

The inventive agents according to this embodiment comprise the substantive dyestuffs preferably in a quantity of 0.01 to 20 wt. %, based on the total colorant.

In addition, the inventive preparations can also comprise naturally occurring dyestuffs contained in henna red, henna neutral, henna black, camomile leaves, sandalwood, black tea, alder buckthorn bark, sage, logwood, madder root, cachou, cedar and alkanet root.

It is not required that the compounds A or 2 or the substantive dyestuffs be each pure compounds. In fact, the inventive hair colorants, due to the manufacturing processes for the individual dyestuffs, may comprise minor quantities of even more components, in so far as they have no detrimental influence on the coloration result or that they must be excluded on other grounds, e.g., toxicological.

In regard to the useable dyestuffs in the inventive hair colorants and hair tints, reference is expressly made to the monograph of Ch. Zviak, The Science of Hair Care, chapter 7 (pages 248-250; substantive dyes) and chapter 8, pages 264-267; oxidation dyestuff precursors), published as volume 7 in the series “Dermatology” (Editor: Ch. Culnan and H. Maibach), Verlag Marcel Dekker Inc., New York, Basel, 1986, as well as the “Europäische Inventar der Kosmetik-Rohstoffe,” published by the European Union, obtainable in disk form from the Bundesverband Deutscher Industrie und Handelsunternehmen für Arzneimittel, Reformwaren und Körperpflegemittel e.V., Mannheim.

The inventive dyes can furthermore comprise all active substances, additives and auxiliaries known for such preparations. In many cases the dyes comprise at least one surfactant, wherein, in principal, not only anionic, but also zwitterionic, ampholytic, nonionic and cationic surfactants are suitable. However, in many cases it has proved advantageous to select the surfactants from among anionic, zwitterionic or nonionic surfactants.

Suitable anionic surfactants for the inventive preparations are all anionic surface-active materials that are suitable for use on the human body. They are characterized by a water solubilizing anionic group, such as e.g., a carboxylate, sulfate, sulfonate or phosphate group and a lipophilic alkyl group containing about 10 to 22 carbon atoms. In addition, the molecule may contain glycol or polyglycol ether groups, ester, ether and amide groups as well as hydroxyl groups. Exemplary suitable anionic surfactants are, each in the form of the sodium, potassium and ammonium as well as the mono-, di- and trialkanol ammonium salts with 2 or 3 carbon atoms in the alkanol group,
linear fatty acids containing 10 to 22 carbon atoms (soaps),
ether carboxylic acids of the formula R—O—(CH₂—CH₂O)ₓ—CH₂—COOH, in which R is a linear alkyl group with 10 to 22 carbon atoms and x=0 or 1 to 16,
acyl sarcosides with 10 to 18 carbon atoms in the acyl group,
acyl taurides with 10 to 18 carbon atoms in the acyl group,
acyl isethionates with 10 to 18 carbon atoms in the acyl group,
sulfosuccinic acid mono- and dialkyl esters with 8 to 18 carbon atoms in the alkyl group and sulfosuccinic acid mono-alkyl polyoxyethyl esters with 8 to 18 carbon atoms in the alkyl group and 1 to 6 oxyethylene groups,
linear alkane sulfonates with 12 to 18 carbon atoms,
linear alpha-olefin sulfonates with 12 to 18 carbon atoms
alpha-sulfo fatty acid methyl esters of fatty acids with 12 to 18 carbon atoms,
alkyl sulfates and alkyl polyglycol ether sulfates of formula R—O(CH₂—CH₂O)ₓ—SO₃H, in which R is preferably a linear alkyl group with 10 to 18 carbon atoms and x=0 or 1 to 12,
mixtures of surface active hydroxysulfonates according to DE-A-37 25 030,
sulfated hydroxyalkyl polyethylene- and/or hydroxyalkylene propylene glycol ethers according to DE-A-37 23 354,
sulfonated unsaturated fatty acids with 12 to 24 carbon atoms and 1 to 6 double bonds according to DE-A-39 26 344 and
esters of tartaric acid and citric acid with alcohols, which represent the addition products of about 2-15 molecules of ethylene oxide and/or propylene oxide on fatty alcohols with 8 to 22 carbon atoms.

Preferred anionic surfactants are alkyl sulfates, alkyl polyglycol ether sulfates and ether carboxylic acids with 10 to 18 carbon atoms in the alkyl group and up to 12 glycol ether groups in the molecule, and especially salts of saturated and particularly unsaturated C₈-C₂₂ carboxylic acids, such as oleic acid, stearic acid, isostearic acid and palmitic acid.

Nonionic surfactants comprise e.g., a polyol group, a polyalkylene glycol ether group or a combination of polyol and polyglycol ether groups as the hydrophilic group. Exemplary compounds of this type are
Addition products of 2 to 30 moles ethylene oxide and/or 0 to 5 moles propylene oxide to linear fatty alcohols with 8 to 22 carbon atoms, to fatty acids with 12 to 22 carbon atoms and to alkyl phenols with 8 to 15 carbon atoms in the alkyl group,
C₁₂-C₂₂ fatty acid mono and diesters of addition products of 1 to 30 moles ethylene oxide on glycerine,
C₈-C₂₂ alkyl mono- and oligoglycosides and their ethoxylated analogs and
Addition products of 5 to 60 moles ethylene oxide on castor oil and hydrogenated castor oil.

Preferred nonionic surfactants are alkyl polyglycosides of the general formula R¹O-(Z)ₓ. These compounds are characterized by the following parameters.

The alkyl group R¹ comprises 6 to 22 carbon atoms and may be both linear and also branched. Primary linear aliphatic groups and aliphatic groups that are methyl-branched in the 2-position, are preferred. Such alkyl groups are, for example, 1-octyl, 1-decyl, 1-lauryl, 1-myristyl, 1-cetyl and 1-stearyl. 1-Octyl, 1-decyl, 1-lauryl, 1-myristyl are particularly preferred. On using so-called “oxo alcohols” as starting materials, compounds with an odd number of carbon atoms in the alkyl chain predominate.

The alkyl polyglycosides used according to the invention may simply comprise, for example, a defined alkyl group R¹. However normally, these compounds are manufactured from natural fats and oils or mineral oils. In which case, the alkyl groups R are present as mixtures corresponding to the starting compounds or to each of the compounds worked up.

Such alkyl polyglycosides are particularly preferred in which R¹ consists
essentially of C₈- and C₁₀ alkyl groups,
essentially of C₁₂- and C₁₄ alkyl groups,
essentially of C₈- to C₁₆ alkyl groups or
essentially of C₁₂- to C₁₆ alkyl groups.

Any mono or oligosaccharide can be employed as the sugar building block Z. Usually, sugars with 5 or 6 carbon atoms as well as the corresponding oligosaccharides are used. Such sugars are, for example, glucose, fructose, galactose, arabinose, ribose, xylose, lyxose, allose, altrose, mannose, gulose, idose, talose and sucrose. Preferred sugar building blocks are glucose, fructose, galactose, arabinose and sucrose; glucose is particularly preferred.

The alkyl polyglycosides used according to the invention comprise on average 1.1 to 5 sugar units. Alkyl polyglycosides with x-values of 1.1 to 1.6 are preferred. Alkyl polyglycosides with x-values of 1.1 to 1.4 are quite particularly preferred.

In addition to their surfactant effect, the alkyl glycosides also serve to improve the fixing of fragrant components on the hair. Thus, when it is desirable for the effect of the perfume oil on the hair to last beyond the hair treatment, the person skilled in the art will preferably have recourse to this class of substances as a further ingredient of the inventive preparations.

The alkoxylated homologs of the cited alkyl polyglycosides can also be used according to the invention. These homologs can comprise on average up to 10 ethylene oxide and/or propylene oxide units per alkyl glycoside unit.

Moreover, zwitterionic surfactants can be used, particularly as co-surfactants. Zwitterionic surfactants are designated as those surface-active compounds that carry at least one quaternary ammonium group and at least one —COO⁻ or —SO₃⁻ group in the molecule. Particularly suitable zwitterionic surfactants are the so-called betaines such as the N-alkyl-N,N-dimethyl ammonium glycinates, for example, the cocoalkyl dimethyl ammonium glycinate, N-acylaminopropyl-N,N-dimethyl ammonium glycinates, for example, the cocoacylaminopropyl dimethyl ammonium glycinate, and 2-alkyl-3-carboxymethyl-3-hydroxyethyl imidazolines with 8 to 18 carbon atoms in each of the alkyl or acyl groups, as well as cocoacylaminoethyl hydroxyethyl carboxymethyl glycinate. A preferred zwitterionic surfactant is the fatty acid amide derivative, known under the INCI name cocamidopropyl betaine.

Likewise suitable, in particular as co-surfactants, are ampholytic surfactants. The ampholytic surfactants are understood to include such surface-active compounds that apart from a C₈₋₁₈ alkyl or acyl group, comprise at least one free amino group and at least one COOH or SO₃H group in the molecule, and are able to form internal salts. Examples of suitable ampholytic surfactants are N-alkylglycines, N-alkyl propionic acids, N-alkylamino butyric acids, N-alkylimino propionic acids, N-hydroxyethyl-N-alkylamidopropylglycines, N-alkyltaurines, N-alkylsarcosines, 2-alkylamino propionic acids and alkylamino acetic acids, each with about 8 to 18 carbon atoms in the alkyl group. Particularly preferred ampholytic surfactants are N-cocoalkylamino propionate, the cocoacylaminoethylamino propionate and the C₁₂₋₁₈ acyl sarcosine.

According to the invention, surfactants of the type quaternary ammonium compounds, esterquats and the amido amines are particularly employed as the cationic surfactants.

Preferred quaternary ammonium compounds are ammonium halides, particularly chlorides and bromides, such as alkyl trimethyl ammonium chlorides, dialkyl dimethyl ammonium chlorides and trialkyl methyl ammonium chloride, e.g., cetyl trimethyl ammonium chloride, stearyl trimethyl ammonium chloride, distearyl dimethyl ammonium chloride, lauryl dimethyl ammonium chloride, lauryl dimethyl benzyl ammonium chloride and tricetyl methyl ammonium chloride, as well as the imidazolium compounds known under the INCI designations Quaternium-27 and Quaternium-83. The long alkyl chains of the abovementioned surfactants have preferably 10 to 18 carbon atoms.

Esterquats are known compounds, which both comprise at least one ester function and also a quaternary ammonium group as structural elements. Preferred esterquats are quaternized ester salts of fatty acids with triethanolamine, quaternized ester salts of fatty acids with diethanolalkylamines and quaternized ester salts of fatty acids with 1,2-dihydroxypropyldialkylamines. Such products are marketed, for example, under the trade names Stepantex®, Dehyquart® and Armocare®. The products Armocare® VGH-70, an N,N-bis(2-palmitoyloxyethyl)dimethyl ammonium chloride, as well as Dehyquart® F-75, and Dehyquart® AU-35 are examples of such esterquats.

The alkylamido amines are normally manufactured by the amidation of natural or synthetic fatty acids and fatty acid fractions with dialkylamino amines. According to the invention, a particularly suitable compound from this substance group is represented by stearamidopropyldimethylamine, commercially available under the designation Tegamid® S 18.

The quaternized protein hydrolyzates illustrate further inventively usable cationic surfactants.

Cationic silicone oils, such as, for example, the commercially available products Q2-7224 (manufacturer: Dow Corning; a stabilized trimethylsilylamodimethicone), Dow Corning® 929 emulsion (comprising a hydroxylamino modified silicone, also referred to as amodimethicone), SM-2059 (manufacturer: General Electric), SLM-55067 (manufacturer: Wacker), and Abil®-Quat 3270 and 3272 (manufacturer: Th. Goldschmidt; diquaternary polydimethylsiloxanes, Quaternium-80) are similarly suitable according to the invention.

An example of a suitable cationic surfactant quaternary sugar derivative is the commercial product Glucquat®100, a “lauryl methyl gluceth-10 hydroxypropyl dimonium chloride” according to INCI nomenclature.

For compounds with alkyl groups that are used as surfactants, they may each be pure substances. However, it is normally preferred to start with natural vegetal or animal raw materials for the manufacture of these materials, with the result that mixtures of substances are obtained, which have different alkyl chain lengths that depend on each raw material.

For surfactants, which are represented by the addition products of ethylene oxide and/or propylene oxide to fatty alcohols or derivatives of these addition products, both products with a “normal” homolog distribution as well as those with a narrow homolog distribution may be used. The term “normal” homolog distribution is understood to mean mixtures of homologs obtained from the reaction of fatty alcohols and alkylene oxide using alkali metals, alkali metal hydroxides or alkali metal alcoholates as catalysts. On the other hand, narrow homolog distributions are obtained if e.g., hydrotalcite, alkaline earth metal salts of ether carboxylic acids, alkaline earth metal oxides, hydroxides or alcoholates are used as catalysts. The use of products with a narrow homolog distribution can be preferred.

Furthermore, the inventive dyes can comprise additional active substances, auxiliaries and additives, such as, for example,
nonionic polymers, such as, for example, vinyl pyrrolidone/vinyl acrylate copolymers, polyvinyl pyrrolidone and vinyl pyrrolidone/vinyl acetate copolymers and polysiloxanes,
cationic polymers, such as quaternized cellulose ethers, polysiloxanes with quaternary groups, dimethyl diallyl ammonium chloride polymers, acrylamide-dimethyl diallyl ammonium chloride copolymers, dimethylaminoethyl methacrylate-vinyl pyrrolidone copolymers quaternized with diethyl sulfate, vinyl pyrrolidone-imidazolinium methochloride copolymers and quaternized polyvinyl alcohol,
zwitterionic and amphoteric polymers such as, for example, acrylamidopropyl-trimethylammonium chloride/acrylate copolymers and octylacrylamide/methyl methacrylate/tert-butylaminoethyl methacrylate/2-hydroxypropyl methacrylate copolymers,
anionic polymers, such as, for example, polyacrylic acids, crosslinked polyacrylic acids, vinyl acetate/crotonic acid copolymers, vinyl pyrrolidone/vinyl acrylate copolymers, vinyl acetate/butyl maleate/isobornyl acrylate copolymers, methyl vinyl ether/maleic anhydride copolymers and acrylic acid/ethyl acrylate/N-tert-butylacrylamide terpolymers,
amphiphilic polymers, such as, for example, the polymers with the INCI designation: designations Acrylates/Beheneth-25 Methacrylate Copolymer, Acrylates/C 10-30 Alkyl Acrylate Crosspolymer, Acrylates/Ceteth-20 Itaconate Copolymer, Acrylates/Ceteth-20 Methacrylate Copolymer, Acrylates/Laureth-25 Methacrylate Copolymer, Acrylates/Palmeth-25 Acrylate Copolymer, Acrylates/Palmeth-25 Itaconate Copolymer, Acrylates/Steareth-50 Acrylate Copolymer, Acrylates/Steareth-20 Itaconate Copolymer, Acrylates/Steareth-20 Methacrylate Copolymer, Acrylates/Stearyl Methacrylate Copolymer, Acrylates/Vinyl Isodecanoate Crosspolymer,
thickeners like agar-agar, guar gum, alginates, xanthane gum, gum arabica, karaya gum, locust bean flour, linseed gums, dextrans, cellulose derivatives, e.g., methyl cellulose, hydroxyalkyl cellulose and carboxymethyl cellulose, starch fractions and derivatives of amylose, amylopectin and dextrins, clays such as e.g., bentonite or synthetic hydrocolloids such as e.g., polyvinyl alcohol,
structurants such as maleic acid and lactic acid,
hair conditioning compounds like phospholipids, for example, soya lecithin, egg lecithin and cephalin,
protein hydrolyzates, particularly those of elastin, collagen, keratin, milk protein, soya protein and wheat protein, their condensation products with fatty acids as well as quaternized protein hydrolyzates,
perfume oils, dimethyl isosorbitol and cyclodextrins,
solvents and solubilizers such as ethanol, isopropanol, ethylene glycol, propylene glycol, glycerine and diethylene glycol,
fiber structure improvers, particularly mono, di and oligosaccharides, such as, for example, glucose, galactose, fructose, fruit sugar and lactose,
quaternized amines, such as methyl 1-alkylamidoethyl-2-alkylimidazolium methosulfate
defoamers such as silicones,
dyestuffs to color the composition,
anti-dandruff active materials like piroctone olamine, zinc omadine and climbazole,
photo protective agents, in particular, derivatized benzophenones, cinnamic acid derivatives and triazines,
substances for adjusting the pH, such as, for example, customary acids, in particular, food acids and bases,
active ingredients, such as allantoin, pyrrolidone carboxylic acids and salts thereof, and bisabolol,
vitamins, provitamins and vitamin precursors, in particular, those of groups A₁, B₃, B₅, B₆, C, E, F and H,
plant extracts such as extracts from green tea, oak bark, stinging nettle, hamamelis, hops, henna, camomile, burdock root, field horsetail, hawthorn, linden flowers, almonds, aloe vera, spruce needles, horse chestnut, sandal wood, juniper, coconut, mango, apricot, lime, wheat, kiwi, melon, orange, grapefruit, sage, rosemary, birch, malva, lady's smock, common yarrow, thyme, lemon balm, rest-harrow, coltsfoot, marshmallow (althaea), meristem, ginseng and ginger,
cholesterol,
thickeners like sugar esters, polyol esters or polyol alkyl ethers,
fats and waxes like spermaceti, beeswax, montan wax and paraffins, fatty acid alkanolamides,
chelating agents like EDTA, NTA, β-alanine diacetic acid and phosphonic acids,
swelling and penetration agents such as glycerol, propylene glycol monoethyl ether, carbonates, hydrogen carbonates, guanidines, ureas, and primary, secondary and tertiary phosphates,
opacifiers such as latex, styrene/PVP copolymers and styrene/acrylamide copolymers,
pearlizing agents such as ethylene glycol mono- and distearate as well as PEG-3-distearate,
pigments,
stabilizers for hydrogen peroxide and other oxidizing agents,
blowing agents such as propane-butane mixtures, N₂O, dimethyl ether, CO₂ and air, and
antioxidants.

With regard to further optional ingredients and their amounts used, reference is expressly made to the relevant handbooks known to the expert, for example, the monograph by K. Schrader, Grundlagen und Rezepturen der Kosmetika, 2nd edition, Hüthig Buch Verlag, Heidelberg, 1989.

According to the invention, however, the dyeing composition can also be applied to the hair together with an oxidation activator that activates the oxidation of the dyestuff precursors by atmospheric oxygen. The oxidation activators are preferably selected from the group formed from carbonates, hydrogen carbonates, carbamates, carboxylic acid esters or their salts, aldehydes, particularly aliphatic aldehydes, 1,3-dihydroxyacetone, imidazole and its derivatives, alkali metal and ammonium peroxydisulfates, metal ions, iodides, quinones and enzymes.

As the oxidative coloration is developed by atmospheric air, it can be inventively advantageous to use metal ions as the oxidation activator.

Suitable metal ions are, for example, Zn²⁺, Cu²⁺, Fe²⁺, Fe³⁺, Mn²⁺, Mn⁴⁺, Li⁺, Mg²⁺, Ca²⁺ and Al³⁺, Zn²⁺, Cu²⁺ and Mn²⁺ are particularly suitable here. In principle, the metal ions can be employed in the form of any physiologically compatible salt or in the form of a complex compound. Preferred salts are the acetates, sulfates, halides, lactates and tartrates. By use of these metal salts, both the formation of the coloration can be accelerated, and the color tint can be selectively influenced.

The activators are preferably comprised in the inventive agents in amounts of 0.01 to 5 wt. %, based on the total dye.

The application temperatures can be in a range between 15 and 40° C. After a contact time of generally 5 to 45 minutes, the hair dye is removed from the hair by rinsing. There is no need to wash the hair with a shampoo if a strong surfactant-containing carrier, e.g., a color enhancing shampoo, was used.

A second subject matter of the present invention is a method for dyeing keratinic fibers, in which an inventive hair dye is applied to the fibers and rinsed out again after a contact time.

### EXAMPLES

The following inventive dye formulations E1 to E6 (see Table 1) were prepared by means of a preparative method known to the person skilled in the art. The following raw materials were used:

TABLE 1

| | E1 | E2 | E3 | E4 | E5 | E6 |
|---|---|---|---|---|---|---|
| Raw materials: | wt. % | wt. % | wt. % | wt. % | wt. % | wt. % |
| Hydrenol ® D | 8.0 | 8.0 | 8.0 | 8.0 | 8.0 | 8.0 |
| Lorol ® techn. | 2.4 | 2.4 | 2.4 | 2.4 | 2.4 | 2.4 |
| Eumulgin ® B 1 | 0.5 | 0.5 | 0.5 | 0.5 | 0.5 | 0.5 |
| Eumulgin ® B 2 | 0.5 | 0.5 | 0.5 | 0.5 | 0.5 | 0.5 |
| Akypo ® Soft 45 NV | 10.0 | 10.0 | 10.0 | 10.0 | 10.0 | 10.0 |
| Texapon ® K 14 S | 2.8 | 2.8 | 2.8 | 2.8 | 2.8 | 2.8 |
| Plantacare ® 1200 UP | 2.0 | 2.0 | 2.0 | 2.0 | 2.0 | 2.0 |
| Eutanol ® G | 1.0 | 1.0 | 1.0 | 1.0 | 1.0 | 1.0 |
| Ascorbic acid | 0.2 | 0.2 | 0.2 | 0.2 | 0.2 | 0.2 |
| (a) | | | | | | |
| 5,6-Dihydroxyindoline•HBr | 1.92 | 1.92 | 0.95 | 1.6 | 1.6 | 1.9 |
| (b) | | | | | | |
| 2-(2-Hydroxyethyl)-p-phenylenediamine H₂SO₄ | 0.6 | — | — | — | — | 0.6 |
| 2,4,5,6-Tetraaminopyrimidin•H₂SO₄, | — | 0.36 | 0.42 | — | 0.36 | — |
| (c) | | | | | | |
| 1,3-Dihydroxynaphthalene | — | — | — | — | 0.7 | — |
| 1,5-Dihydroxynaphthalene | — | — | 0.5 | — | — | 0.5 |
| 2-Methylresorcinol | 0.6 | 0.79 | 0.79 | 0.5 | — | 0.6 |
| 2-Amino-3-hydroxypyridine | 0.26 | — | — | — | — | — |
| (d) | | | | | | |
| AMP ® 95 | — | — | — | 3.6 | — | — |
| Potassium hydroxide (50 wt. % solution in water | 2.3 | 2.05 | 2.3 | — | 2.3 | 2.3 |
| Monoethanolamine | — | — | — | — | 0.05 | — |
| Perfume | 0.3 | 0.3 | 0.3 | 0.3 | 0.3 | 0.3 |
| Water | ad 100 | ad 100 | ad 100 | ad 100 | ad 100 | ad 100 |

A strand of natural hair was colored with each agent. In each case the hair acquired an intensive, long lasting natural coloration without red, blue or violet tinges.

1. Agent for atmospheric oxygen-induced dyeing of keratinic fibers, especially human hair, comprising a cosmetically acceptable carrier and
(a) at least one derivative of indole and/or indoline as the dyestuff precursor of a nature-analogous dyestuff,
(b) at least one oxidation dyestuff precursor of the developer type,
(c) at least one oxidation dyestuff precursor of the coupler type and
(d) at least one alkalization agent, with the proviso that
the agent is free of ammonia and
the agent is free of additional oxidizing agents capable of oxidizing components (a), (b) and (c).

2. The agent of claim 1 wherein the indoline derivative is a compound of the formula (1a) and/or a physiologically compatible salt of a compound of the formula (1a) with an organic or inorganic acid 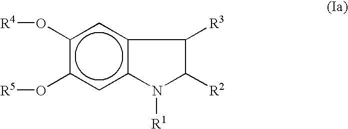
wherein
R¹ is hydrogen, a C₁-C₄-alkyl group or a C₁-C₄-hydroxyalkyl group,
R² is hydrogen or a —COOH group, where the —COOH group may also be present as the salt with a physiologically compatible cation,
R³ is hydrogen or a C₁-C₄-alkyl group,
each of R⁴ and R⁵ is independently hydrogen, a C₁-C₄-alkyl group or a —CO—R⁶ group, wherein R⁶ is a C₁-C₄-alkyl group.

3. The agent of claim 1 wherein the indole derivative is a compound of the formula (1b) and/or a physiologically compatible salt of a compound of the formula (1b) with an organic or inorganic acid 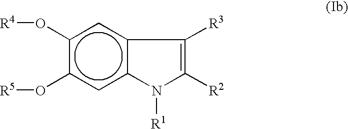
wherein
R¹ is hydrogen, a C₁-C₄-alkyl group or a C₁-C₄-hydroxyalkyl group,
R² is hydrogen or a —COOH group, where the —COOH group may also be present as the salt with a physiologically compatible cation,
R³ is hydrogen or a C₁-C₄-alkyl group,
each of R⁴ and R⁵ is independently hydrogen, a C₁-C₄-alkyl group or a —CO—R⁶ group, wherein R⁶ is a C₁-C₄ alkyl group.

4. The agent of claim 1 wherein the amount of the dyestuff precursor of the nature-analogous dyestuff (a) is from 0.01 to 10 wt. %, based on the weight of the agent.

5. The agent of claim 1 wherein the oxidation dyestuff precursor of the developer type (b) is selected from the group consisting of p-phenylenediamine derivatives, binuclear developer components, p-amino phenol and its derivatives, pyrimidine derivatives, pyrazole derivatives as well as pyrazolopyrimidine derivatives and the physiologically compatible salts of these compounds.

6. The agent of claim 1 wherein the amount of the dyestuff precursor (b) is from 0.01 to 5 wt. %, based on the weight of the agent.

7. The agent of claim 1 wherein the oxidation dyestuff precursor of the coupler type (c) is selected from the group consisting of m-amino phenol and its derivatives, o-amino phenol and its derivatives, m-phenylenediamine and its derivatives, o-phenylenediamine and its derivatives, dihydroxybenzene derivatives, trihydroxybenzene derivatives, pyridine derivatives, naphthalene derivatives, morpholine derivatives, quinoxaline derivatives, pyrazolone derivatives and methylenedioxybenzene derivatives.

8. The agent of claim 1 wherein the amount of the dyestuff precursor of the coupler type (c) is from 0.01 to 5 wt. %, based on the weight of the agent.

9. The agent of claim 1 wherein the mole ratio of the dyestuff precursor of the nature-analogous dyestuffs (a) to the developer components (b) is from 10 to 1 to 1 to 2.

10. The agent of claim 9 wherein the mole ratio is from 6 to 1 to 3 to 1.

11. The agent of claim 1 wherein the mole ratio of the coupler components (c) to the developer components (b) is from 8 to 1 to 1 to 2.

12. The agent of claim 11 wherein the mole ratio is from 2.5 to 1 to 4.5 to 1.

13. The agent of claim 1 wherein the mole ratio of the nature-analogous dyestuffs (a) to the coupler components (c) is from 2 to 1 to 1 to 2.

14. The agent of claim 13 wherein the mole ratio is from 1.5 to 1 to 1 to 1.

15. The agent of claim 1 wherein the alkalization agent (d) is selected from the group consisting of basic amino acids, alkali metal hydroxides, alkanolamines, alkali metal carbonates, alkali metal hydrogen carbonates, alkali metal metasilicates, alkali metal phosphates and alkali metal hydrogen phosphates.

16. The agent of claim 1 wherein pH is in the range pH 6 to 12.

17. The agent of claim 1 further comprising a surfactant selected from the group consisting of anionic surfactants, zwitterionic surfactants, ampholytic surfactants, nonionic surfactants and cationic surfactants.

18. The agent of claim 1 further comprising at least one polymer selected from the group consisting of anionic polymers, cationic polymers, amphoteric polymers and amphiphilic polymers.

19. Method of dyeing keratinic fibers comprising contacting the fibers with an effective amount of an agent of claim 1 and rinsing the fibers after a contact time sufficient to dye the fibers.
